(19) United States
(12) Patent Application Publication
(10) Pub. No.: US 2002/0064770 A1
(43) Pub. Date: May 30, 2002

(54) Binding compounds and methods for identifying binding compounds

(76) Inventors: John J. Nestor JR., Bedford, MA (US); Carol J. Wilson, Somerville, MA (US); Raymond H. See, Boston, MA (US); Christina A. Tan Hehir, Boston, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/813,653

(22) Filed: Mar. 20, 2001

Related U.S. Application Data
(60) Provisional application No. 60/190,946, filed on Mar. 21, 2000.
(60) Provisional application No. 60/190,996, filed on Mar. 21, 2000.
(60) Provisional application No. 60/191,299, filed on Mar. 21, 2000.

(51) Int. Cl. C12Q 1/70; C12Q 1/68; G01N 33/53; G01N 33/543

(52) U.S. Cl. 435/5; 435/6; 435/7.1; 436/518

(57) ABSTRACT

Compositions of binding compounds for CC chemokine receptor 5 and methods for identifying binding compounds for CC chemokine 5 receptor are provided. Also provided are therapeutic agents comprising such compounds.

### RELATED APPLICATIONS

This application claims the benefit of U.S. Ser. No. 60/190,946, U.S. Ser. No. 60/190,996 and U.S. Ser. No. 60/191,299, the disclosure of each of which is incorporated by reference herein. Reference is also made to the applications filed on Mar. 20, 2001 identified by attorney docket nos. CNS-006 and CNS-007, the disclosure of each of which is incorporated by reference herein.

### FIELD OF THE INVENTION

The invention relates generally to Cysteine-Cysteine Chemokine Receptor 5 (“CCR5” or “CC chemokine receptor 5”), and more particularly, to binding compounds for CC chemokine receptor 5. Methods of the invention are useful for the treatment of disease by identifying and preparing therapeutic binding compounds for CC chemokine receptor 5.

### BACKGROUND OF THE INVENTION

Chemokines (chemoattractant cytokines) comprise a family of structurally related secreted proteins of about 70-110 amino acids that share the ability to induce migration and activation of specific types of blood cells. See Proost P., et al. (1996) Int. J. Clin. Lab. Rse. 26: 211-223; Premack, et al. (1996) Nature Medicine 2: 1174-1178; Yoshie, et al. (1997) J. Leukocyte Biol. 62: 634-644. Over 30 different human chemokines have been described to date. While they are primarily responsible for the activation and recruitment of leukocytes, they vary in their specificities for different leukocyte types (neutrophils, monocytes, eosinophils, basophils, lymphocytes, dendritic cells, etc.), and in the types of cells and tissues where the chemokines are synthesized. Further analysis of this family of proteins has shown that it can be divided up into two further subfamilies of proteins. These have been termed CXC or α-chemokines, and the CC or β-chemokines based on the spacings of two conserved cysteine residues near the amino terminus of the proteins.

Chemokines are typically produced at sites of tissue injury or stress, where they promote the infiltration of leukocytes into tissues and facilitate an inflammatory response. Some chemokines act selectively on immune system cells such as subsets of T-cells or B lymphocytes or antigen presenting cells, and may thereby promote immune responses to antigens. In addition, some chemokines have the ability to regulate the growth or migration of hematopoietic progenitor and stem cells that normally differentiate into specific leukocyte types, thereby regulating leukocyte numbers in the blood.

The activities of chemokines are mediated by cell surface receptors that are members of a family of seven transmembrane (“7TM”), G-protein coupled receptors (“GPCR”). At least twelve different human chemokine receptors are known, including CCR1, CCR2, CCR3, CCR4, CCR5, CCR6, CCR7, CCR8, CXCR1, CXCR2, CXCR3, and CXCR4. These receptors vary in their specificities for specific chemokines. Some receptors bind to a single known chemokine, while others bind to multiple chemokines. Binding of a chemokine to its receptor typically induces intracellular signaling responses such as a transient rise in cytosolic calcium concentration, followed by cellular biological responses such as chemotaxis. In addition, some chemokine receptors, such as CCR5, serve as co-receptors for Human Immunodeficiency Virus (HIV), such that they interact with HIV and with the cellular CD4 receptor to facilitate viral entry into cells.

Chemokines are important in medicine because they regulate the movement and biological activities of leukocytes in many disease situations, including, but not limited to: allergic disorders, autoimmune diseases, ischemia/reperfusion injury, development of atherosclerotic plaques, cancer (including mobilization of hematopoietic stem cells for use in chemotherapy or myeloprotection during chemotherapy), chronic inflammatory disorders, chronic rejection of transplanted organs or tissue grafts, chronic myelogenous leukemia, and infection by HIV and other pathogens. Furthermore, CCR5, in particular, has been implicated in diseases such as multiple sclerosis and rheumatoid arthritis. See, e.g., Balashov et al., Proc. Natl. Acad. Sci., USA 96:6873-6878 (1999); and Mack, et al., Arthritis Rheum. 42(5):981-8 (1999).

Antagonists of chemokine receptors may be of benefit in many of these diseases by reducing excessive inflammation and immune system responses. In the case of HIV infection, chemokines and antagonists that bind to HIV co-receptors may have utility in inhibiting viral entry into cells. HIV causes Acquired Immune Deficiency Syndrome (“AIDS”), which is one of the leading causes of death in the United States and throughout the world. According to the Center for Disease Control, at least 30.6 million people world-wide have been infected with HIV. HIV attacks the immune system and leaves the body vulnerable to a variety of life-threatening illnesses. Common bacteria, yeast, and viruses that would not cause disease in people with a fully functional immune system often cause these illnesses in people affected with HIV.

Not all patients infected with HIV have AIDS. Typically, a patient who has been infected with HIV will slowly develop AIDS as HIV damages his immune system. The severity of the immune system damage is measured by an absolute CD4⁺ lymphocyte count; a patient having a count of less than 200 cells/μl is considered to have AIDS. The CD4 protein is a glycoprotein of approximately 60,000 molecular weight and is expressed on the cell membrane of mature, thymus-derived (T) lymphocytes, and to a lesser extent on cells of the monocyte/macrophage lineage. Typically, CD4 cells appear to function by providing an activating signal to B cells, by inducing T lymphocytes bearing the reciprocal CD8 marker to become cytotoxic/suppressor cells, and/or by interacting with targets bearing major histocompatibility complex (MHC) class II molecules.

The search for a preventative or therapeutic agent for HIV and AIDS has been especially intense as this epidemic has proliferated world-wide. Research has discovered that the ability of HIV to enter cells requires the binding of the HIV envelope glycoproteins encoded by the env gene to the CD4 receptor. These glycoproteins are encoded by the env gene and translated as a precursor, gp160, which is subsequently cleaved into gp120 and gp41. Gp120 binds to the CD4 protein present on the surface of susceptible target cells, resulting in the fusion of virus with the cell membranes, and facilitating virus entry into the host. The eventual expression of env on the surface of the HIV-infected host cell enables this cell to fuse with uninfected, CD4⁺ cells, thereby spreading the virus. However, in response to infection with HIV, the host immune system will produce antibodies targeted against various antigenic sites, or determinants, of gp120. Some of those antibodies will have a neutralizing effect and will inhibit HIV infectivity. It is believed that this neutralizing effect is due to the antibodies' ability to interfere with HIV's cellular attachment. It is also believed that this effect may explain in part, the rather long latency period between the initial seroconversion and the onset of clinical symptoms.

Recent studies have shown that the HIV fusion process occurs with a wide range of human cell types that either express human CD4 endogenously or have been engineered to express human CD4. The fusion process, however, does not occur with nonhuman cell types engineered to express human CD4. Although such nonhuman cells can still bind env, membrane fusion does not follow. The disparity between human and nonhuman cell types exists apparently because membrane fusion requires the co-expression of human CD4 and a co-receptor specific to human cell types. Because they lack this co-receptor accessory factor, nonhuman cell types engineered to express only human CD4 are incapable of membrane fusion, and are thus nonpermissive for HIV infection. Furthermore, expression of CD4 in some human cell lines was insufficient to confer resistance to HIV-1 infection. In addition, some HIV-1 strains were T cell tropic (T-tropic) while others were macrophage tropic (M-tropic), though both cells possessed the CD4 antigen. Further research has shown that certain chemokines could block the infectivity of M-tropic but not T-tropic HIV strains. Thereafter, it was shown that a particular receptor was required for the activity of T-tropic strains. See e.g., Horuk R., Immunol Today 20(2):89-94 (1999); Doms R W, Peiper S C., Virology 235(2):179-90 (1997); Ward S G, Bacon K, Westwick J., Immunity 9(1):1-11 (1998); Berson J F, Doms R W., Semin Immunol 10(3):237-48 (1998).

While it has been demonstrated that HIV uses the CCR5 as a co-receptor for cellular entry, it has been difficult for researchers to obtain high resolution X-ray crystallographic structures of a CCR5 because of difficulties in crystallizing such a 7TM protein which requires complex interactions with lipids for its native conformation. The requirement of the interaction with lipids also makes difficult the preparation of biologically active forms of such GPCRs, because, in the absence of those lipids, they readily form denatured aggregates with minimal to no ability to specifically bind ligands unless great care is taken to preserve the biologically active conformation during solubilization. In the absence of an X-ray structure, a variety of approaches have been used to define the regions of CCR5 that are involved in gp120 binding and viral uptake. These approaches generally involve comparing results with non-human homologues, chimeric receptors, and point mutants to study the structural requirements for the co-receptor activity of CCR5. It is believed that most residues important for CCR5 binding form a cleft with the side chain contacts from ten amino acids. Important residues are associated with two short sequences of amino acids: residues 419-422 and 437-444. See FIG. 4. CCR5, a 352-amino acid protein, has seven putative transmembrane (“TM”) segments (TM1=residues 32-56, TM2=67-88, TM3=103-124, TM4=144-167, TM5=198-220, TM6=236-257, and TM7=281-301, puatively), an extracellular N-terminus, three extracellular loops and three intracellular loops connecting the transmembrane segments, and an intracellular C-terminus. M-tropic viruses are believed to interact with residues in the extracellular domains of CCR5, with the second loop being the most important. Changes in the N-terminal domain have been well tolerated by M-tropic viruses suggesting that interactions with residues in loops 1-3 are sufficient for entry. See e.g. Doms R W, Peiper S C., Virology 235(2):179-90 (1997).

Individuals who have a homozygous 32 base-pair deletion in the CCR5 gene have been shown to be healthy and highly resistant to HIV infection. See e.g., Doms R W, et al., Virology 235(2): 179-90 (1997) (suggesting that the loss of CCR5 is not generally damaging to health). Individuals who are heterozygous for that deletion have a slowed progression of the disease. See e.g., Moore J P, Science 276(5309):51-2 (1997).

Several inhibitors of CCR5 have been reported. TAK 779 antagonizes the binding of RANTES (regulated on activation, normal T cell expressed and secreted) to CCR5-expressing Chinese hamster ovary cells and blocked CCR5-mediated Ca²⁺ signaling at nanomolar concentrations. The inhibition of CC chemokine receptors by TAK-779 appears to be specific to CCR5. TAK-779 displays highly potent and selective inhibition of R5 HIV-1 replication without showing cytotoxicity. The compound appears to inhibit the replication of R5 HIV-1 clinical isolates as well as a laboratory strain at a concentration of 1.6-3.7 nM in peripheral blood mononuclear cells, but was inactive against a T-cell line-tropic HIV-1. See e.g., Baba M, et al., Proc Natl Acad Sci USA 96(10):5698-703 (1999).

Aminooxypentane (AOP)-RANTES[2-68] is a chemically modified form of the chemokine RANTES. (AOP)-RANTES does not induce chemotaxis and is a subnanomolar antagonist of CCR5 function in monocytes. It potently inhibits the infection of macrophages by M-tropic HIV-1 strains. Thus, activation of cells by chemokines is not a prerequisite for the inhibition of viral uptake and replication. See e.g., Simmons G, et al., Science 276(5310):276-9 (1997). More recently, another chemically modified RANTES has been described as more potent: (NNY)-RANTES[2-68]. See e.g., Howard O M, et al., J Med Chem 41(13):2184-93 (1998).

Further prior research has evaluated a series of ureido analogs of distamycin previously reported to block HIV entry into cells in vitro, for chemokine antagonist activity. One of the distamycin analogs, (NSC 651016), inhibits syncytia formation and cell fusion. 16. See e.g., Howard O M, et al., J Med Chem 41(13):2184-93 (1998).

As a result of the limitations of prior inhibitors of CCR5, a need still remains for effective HIV preventative and therapeutic agents, and methods for identifying candidates thereof. It has been demonstrated that HIV uses the CCR5 as a co-receptor for cellular entry that can be blocked by its natural ligands and this makes a high affinity ligand for CCR5 an important therapeutic target. GPCRs in general, and CCR5 in particular, are very difficult to solubilize and purify because they normally need to fold and be maintained in the presence of the native lipids of the cell membrane. Simple expression and precipitation with antibodies result routinely in denatured aggregates with little or no ability to specifically bind native ligands. Accordingly, there is a need in the art for methods of identifying CCR5 binding compounds and identification of CCR5 binding therapeutics with which to prevent or treat diseases such as AIDS. Such therapeutics may comprise peptides, peptidomimetics, or small molecules that can inhibit natural ligand binding to CCR5. Such methods and compositions are provided herein.

### SUMMARY OF THE INVENTION

The present invention provides binding compounds for CCR5 and methods for identifying those binding compounds. In one embodiment, screening methods are provided to identify binding motifs for CCR5, as well as ligands capable of binding to CCR5. In another embodiment, the invention comprises the design and identification of therapeutic peptides, peptidomimetics, or small molecules suitable for use in the prevention or treatment of HIV and AIDS.

In one embodiment, methods of the invention provide for the synthesis and purification of linear and cyclic peptide libraries useful for screening and identifying a binding motif for CCR5, as well as screening for potential ligands thereof. Methods of the invention provide for the incorporation of unnatural amino acids and amino acids of the D configuration into linear or cyclic peptides for use in such libraries. Libraries comprising peptides having such amino acids demonstrate enhanced binding affinity and duration of action in vivo resulting from resistance to proteolysis.

In a preferred embodiment, the invention provides for the use of highly diverse libraries of peptide (linear and cyclic, natural and unnatural amino acids), peptidomimetic, and small molecule compounds for the lead ligand identification step. Such ligands may be directly or indirectly agonistic or antagonistic to CCR5 binding activity.

In a preferred embodiment, the invention provides for the use of phage display methods for the identification of preliminary motif information, followed by additional rounds of affinity purification with purified receptor preparations and highly diverse libraries of the invention. In a particularly preferred embodiment, phage display technology is combined with the use of cyclic peptide and/or peptidomimetic libraries.

In another embodiment of the invention, computer-aided design technology is used to virtually screen, identify, design, or validate lead compounds for agonistic or antagonistic potential with regard to CCR5 activity. Such technology uses computer-generated, three-dimensional images based upon molecular and structural information of both the CCR5 and the potential binding partners by virtually aligning the protein with the binding partners. In the case of a library designed for computer-aided screening, a great deal of the information necessary for lead optimization is obtained directly from the library design. In one embodiment, potential leads are identified by prior screening of an actual library or through some other means. One embodiment of the invention involves the screening of biologically appropriate drugs that relies on structure based rational drug design. In such cases, a three dimensional structure of the protein (or similar family member), peptide or molecule is determined and potential agonists and/or antagonists are designed with the aid of computer modeling. In a preferred embodiment of the invention, after an appropriate drug is identified, the drug is contacted with CCR5, wherein a binding complex forms between the potential drug and CCR5. Methods of contacting the drug to CCR5 are generally understood by anyone having skill in the art of drug development.

In another embodiment, the present invention provides for the use of partially purified CCR5 receptor protein as the agent for carrying out the selection, identification, and improvement of tight binding ligands in identifying therapeutically useful compounds. In a preferred embodiment, the invention comprises the use of tagging methods to generate a modified CCR5 receptor protein that functions to facilitate purification and identification steps involved in the screening methods. In another embodiment, the invention comprises a nucleic acid sequence corresponding to the receptor CCR5 fused to tag sequences (i.e., GST, FLAG, 6xHis, dual tagged with FLAG-GST, C-MYC, MBP, V5, Xpress, CBP, HA) with appropriate specific protease sites engineered into the vector.

In a particularly preferred embodiment, methods of the invention provide for solubilization and immobilization of CCR5 to facilitate ligand selection methods provided herein. CCR5 may be derived from any source, such as, for example, cell membrane preparations and whole cell preparations. In one embodiment, the invention provides for a method of screening combinatorial libraries directly for general affinity determination using membranes from baculovirus expression systems or any other appropriate expression system. In one embodiment of the invention, partially purified CCR5 is used in carrying out the selection, identification, and improvement of tight binding ligands. In a preferred embodiment, partially purified, tagged CCR5 is used in a sequestered form to screen diverse libraries (focused or highly diverse) for the affinity purification of a tight binding ligand. In a highly preferred embodiment of the invention, the conditions for solubilization and immobilization of the appropriate ligand provides for the use of low salt, such as, for example, low magnesium or calcium concentrations; and no sodium chloride (“NaCl”) (0.0 nM NaCl).

In another embodiment, the invention comprises the step of eluting bound components of the libraries from the immobilized protein with specific N-terminally blocked peptides or other non-sequencable analogs. In yet another embodiment, the invention comprises the step of binding combinatorial libraries to a resin-immobilized protein. In another embodiment, the invention comprises a purified polypeptide with tag sequences, which may be immobilized onto an appropriate affinity resin for assay. A further embodiment comprises the step of releasing or eluting tagged protein with its bound library with specific N-terminally blocked peptides or other non-sequencable analogs. In yet another embodiment, a method of the invention comprises the step of cleaving a tag from a protein of interest using a specific protease (as designed into the protein/vector) after immobilization onto an affinity resin and after the combinatorial library is bound to release the complex.

In yet another embodiment, the target ligand is selected from a linear peptide library, a peptidomimetic library, a cyclic peptide library, or a focused library developed using an initial motif identified by phage display techniques or a library combining any of the foregoing. In another embodiment, a target ligand is eluted from the receptor preparation using a peptide or other ligand, or by using pH change or chaotropic agents, such as urea or guanidine hydrochloride, that can disrupt the hydrogen bonding structure of water and denature proteins in concentrated solutions by reducing the hydrophobic effect. Also contemplated by the invention are ligands for CCR5 identified using the methods disclosed herein. In yet another embodiment of the invention, protein sequencing techniques are used for the determination of the structure of the ligand identified by the affinity purification step.

In another embodiment, the invention comprises therapeutic agents, such as, for example, a small molecule antagonist of CCR5 binding that are identified using methods of the invention appropriate for the treatment of a disease or disorder, such as, for example, HIV infection or AIDS. In another embodiment, a patient infected with HIV is treated with a therapeutic agent comprising a compound identified using methods of the invention or a small molecule antagonist of CCR5 binding. In another embodiment, a patient infected with HIV is treated through the use of combinations of therapeutics that include, for example, CCR5 inhibitors, reverse transcriptase protease inhibitors, and other anti-HIV therapeutics.

A detailed description of certain preferred embodiments of the invention is provided below. Other embodiments of the invention are apparent upon review of the detailed description that follows.

### DESCRIPTION OF THE DRAWINGS

FIG. 1 shows the gp120 residues involved in CCR5 binding.

FIG. 2 shows a peptide library with a fixed, non-degenerate lysine or arginine and eight degenerate positions consisting of eighteen amino acids in approximately equal proportion.

FIG. 3 shows a peptide library screening using binding domains.

FIG. 4 shows a CCR5 cDNA sequence.

FIG. 5 shows a baculovirus transfer vector for CCR5-HIS.

FIG. 6 shows a baculovirus transfer vector for CCR5-FLAG.

FIG. 7 shows a baculovirus transfer vector for CCR5-GST.

FIG. 8a is a chart showing the characterization of saturation binding of [¹²⁵I]-MIP-1β to CCR5 to determine K_{d}.

FIG. 8b is a chart showing the characterization of displacement of [₁₂₅I]-MIP-1β binding to CCR5 by MIP-1α both in membrane preparations and immobilized on the column.

FIG. 9 shows the immobilization of GPCRs for affinity purification from libraries.

FIG. 10 shows the purification of CCR5-GST using glutathione-sepharose.

FIG. 11 shows the screening of peptide libraries using CCR5-containing membranes.

FIG. 12 is a chart showing the % solubilization of CCR5 under exemplary conditions.

### DETAILED DESCRIPTION OF THE INVENTION

Generally, methods of the invention provide for the determination of a binding motif for CCR5. Further, methods of the invention provide for the identification of agonists or antagonists of the interaction of CCR5 with its natural ligand, thereby providing for the identification of therapeutic lead compounds. Methods for library design and synthesis, and library screening that are particularly useful in the invention are described in the following patent and patent applications, the disclosure of each of which is incorporated by reference herein: Cantley et al., U.S. Pat. No. 5,532,167; Cantley, et al., U.S. Ser. No. 08/369,643, filed Dec. 17, 1998; Cantley, et al, U.S. Ser. No. 08/438,673, filed Nov. 12, 1999; Hung-Sen, et al., U.S. Ser. No. 09/086,371, filed May 28, 1998; Hung-Sen, et al, U.S. Ser. No. 08/864,392, filed Jun. 24, 1999; and Lai, et al., U.S. Ser. No. 09/387,590, filed Aug. 31, 1999.

According to the methods of the invention, CCR5 is cloned and expressed, and tested for activity. The CCR5 may be tagged on the C-terminus or on the N-terminus to facilitate the determination of the character of the CCR5's ligand-binding properties. Exemplary tags include, without limitation, 6xHis, FLAG, GST, V5, Xpress, c-myc, HA, CBD, and MBP. The tagged CCR5 is used in screening of libraries comprising, for example, linear and/or cyclic peptides having natural and/or unnatural amino acids, peptidomimetics and/or small molecules. Such peptidomimetics and small molecules may comprise any natural or synthetic compound, composition, chemical, protein, or any combination or modification of any of the foregoing that is used to screen for binding compounds of CCR5.

In one aspect, an oriented degenerate peptide library method useful in methods of the invention uses soluble peptide libraries consisting of one or more amino acids in non-degenerate positions, known or suspected to be important for ligand binding, and eighteen amino acids in approximately equal proportions in degenerate positions. Cysteine and tryptophan may be omitted to avoid certain analytical difficulties on sequencing. Such a library is shown in FIG. 2, where X represents a degenerate position consisting of any of eighteen amino acids and a lysine or arginine is fixed at a non-degenerate position. Furthermore, the selection of arginine or lysine as an orienting residue is based on the fact that basic residues of gp120 are important determinants in binding to CCR5. Another aspect of the invention involves the selection of any amino acid as an orienting residue. In addition, additional residues can be added to the N-terminal of the sequence shown in FIG. 2 because there are often interfering substances present in the first and second sequencing cycles. Additional residues can be added at the C-terminal end to provide amino acids to better anchor the peptide to the filter in the sequencer cartridge.

Another aspect of the invention provides for the use of highly diverse libraries of peptide (linear and cyclic, natural and unnatural amino acids), peptidomimetic, and small molecule compounds for the lead identification step. For example, these ligands can be agonistic or antagonistic in their function on the receptor. Generally, the invention uses partially purified CCR5 as the agent for carrying out the selection, identification, and improvement of tight binding ligands as a route to therapeutically useful compounds. In addition, the invention provides for the development and use of solubilization and immobilization procedures that facilitate efficient ligand selection methods provided herein. Specifically, the optimal conditions for the solubilization and immobilization for efficient ligand selection comprise the use of low salt, such as, for example, low or no magnesium or calcium concentrations, and no NaCl concentrations (0.0 nM NaCl). Ligand selection methods using, for example, inactive, precipitated protein, cell membrane preparations, and whole cell preparations are further provided herein.

In one aspect of the invention, the screening step may comprise phage display technology. Such phage display systems have been used to screen peptide libraries for binding to selected target molecules and to display functional proteins with the potential of screening these proteins for desired properties. More recent improvements of the display approach have made it possible to express enzymes as well as antibody fragments on the bacteriophage surface thus allowing for selection of specific properties by selecting with specific ligands. See e.g., Smith S F, et al., Methods Enzym. 217:228-257 (1993). Phage display methods may be used for the identification of preliminary motif information, and followed by additional rounds of affinity purification with purified receptor preparations of the invention and highly diverse libraries, especially cyclic peptide and peptidomimetic libraries. The phage display methods allow the identification of motifs of natural amino acids. Information derived from phage display can be taken into affinity purification methods using, for example, synthetic libraries containing novel amino acid analogs or cyclic peptides to select ligands that have enhanced pharmaceutical characteristics. The use of initial, secondary and tertiary libraries allows a more complete definition of the specificity of the binding site. Secondary libraries may be sequenced incorporating information from the initial library. With the first library, some degenerate positions may yield high preferences for specific amino acids and these may become non-degenerate positions consisting of the preferred amino acid in a second library. See e.g., Wu R, J Biol Chem 271(27):15934-41 (1996).

Alternatively, or in addition, computer-aided design technology may be used in the screening and/or designing of peptides, peptidomimetics, and small molecules. Together with information such as, for example, the crystal structure of rhodopsin (see e.g., Palczewski, et al, Science 289(5480):739-745 (2000)) along with the sequence of CCR5, transmembrane predictions, and any structural information obtained from mutagenesis studies, computer aided design technology may virtually screen, identify, design and validate potential compounds with regards to their CCR5 activity. Computer programs that may be used to aid in the design of appropriate peptides, peptidomimetics and small molecules include, for example, Dock, Frodo and Insight. An example of a method for screening of biologically appropriate drugs relies on structure based rational drug design. In such cases, a three dimensional structure of the protein, peptide or molecule is determined (or modeled after a close family member) and potential agonists and/or antagonists are designed with the aid of computer modeling. See e.g., Butt et al, Scientific American, December 92-98 (1993); West et al., TIPS, 16:67-74 (1995); Dunbrack et al., Folding & Design, 2:27-42 (1997). After an appropriate drug is identified, the drug is contacted with CCR5, wherein a binding complex forms between the potential drug and CCR5. Methods of contacting the drug to CCR5 are generally understood by anyone having skill in the art of drug development.

The screening step may be performed in solution phase, or with the CCR5 immobilized on affinity columns. In addition to the immobilization of tagged CCR5 using an affinity resin, other forms of sequestration can be used to perform the affinity purification of select ligands from libraries. These include, but are not limited to the following examples. The receptor and bound library components can be separated from non-bound library components using equilibrium dialysis. The tagged receptor can be bound to specific affinity membranes, which are in the form of plates or are separate. The libraries can then be incubated with the membrane and easily washed to remove non-specific binding components. Size exclusion methodology can be used to separate a purified receptor bound library complex from unbound components after pre-incubating the receptor with the library. Additionally, a micellar complex containing the receptor (which may or may not incorporate lipids as well as detergent) can be separated after binding select affinity components from a library by differential centrifugation. Generally, the high affinity ligand can be released using low pH or high salt conditions and the structure identified by sequencing as described herein.

In order to determine those ligands that had the highest affinity to the target receptor, generally, over 200 peptide libraries were screened to determine each library's respective inhibition binding. In general, a greater than 10% inhibition at 100 μM was significant for continued evaluation of the sequence via affinity purification. In additional aspects of the invention, once preferred amino acid residues are identified due to high preference values by CCR5 at the degenerate positions of the library, specific peptides are synthesized by the same methods as employed for library synthesis. In one embodiment of the invention, a high preference value is greater than 1. The value is determined by subtracting the control value from the sample value and dividing by the reference value. In a preferred embodiment of the invention, the preference value is greater than 1.2. In a highly preferred embodiment of the invention, the preference value is greater than 2. After synthesis of the identified peptide sequence, the peptide is purified by, for example, High Performance Liquid Chromatography (“HPLC”) and compositions are confirmed by Matrix-Assisted Laser Desorption Ionization-Time of Flight Mass Spectrometer (“MALDI-TOF MS”) and Edman Sequencing. Generally, relative affinities may be measured by modifying the radiolabel binding assay used in receptor purification.

To enhance the specificity of the motif obtained from the affinity purified peptides, other methods can be used. The bound components of the libraries can be eluted from the immobilized protein with specific N-terminally blocked peptides or other non-sequencable analogs. To avoid the release of minor contaminants from the affinity resin after binding of the library, the release/elution of the tagged CCR5 with its bound library can be accomplished using specific N-terminally blocked peptides or other non-sequencable analogs. This can be done using acetylated FLAG peptide to elute CCR5-FLAG receptor from the resin. Also, the bound peptide may be eluted directly using an N-terminally blocked peptide or ligand, or other non-sequenceable analog using, for example, (AOP)-RANTES. Alternatively, the tag from CCR5 may be cleaved using a specific protease (as designed into the protein/vector; either enterokinase or thrombin) after immobilization onto the affinity resin and after the combinatorial library is bound to release the complex. Finally, libraries can be prescreened for their ability to bind to the receptor (using significantly less protein) by a binding assay using CCR5-containing membranes from, for example, Sf9 (Spodoptera frugiperda) or High Five (Trichoplusia ni) cells (both obtained from Invitrogen) in a single assay or in an array assay. This screening may be performed using CCR5 and a number of linear and cyclic libraries to determine their effectiveness in inhibiting the natural ligand to bind.

Methods of the invention further comprise the design of therapeutic agents comprising peptides, peptidomimetics, and/or small molecules that are antagonistic to CCR5 activity appropriate for the treatment of patients with a disease, such as AIDS. Binding compounds for CCR5 and the identification of optimal synthesis and purification thereof provides for an effective treatment of AIDS and HIV infection. For example, the small peptide ligand binding compounds of the invention, both cyclic and linear peptide ligands, will have enhanced binding affinity and action, and are resistant to proteolysis. Compounds that exhibited enhanced affinity and action include, for example, M-A-R-S-L-I-W-R-P-A-K-A-K-K-A, K-K-K-A-R-S-L-I-W-R-P-A-K-A-K-K-K, and cyclo(M-Y-A-T-R-W-K-N). Amino acids and peptides are abbreviated and designated following the rules of the IUPAC-IUB Commission of Biochemical Nomenclature in J. Biol. Chem. 247, 977-983 (1972). Amino acid symbols denote the L-configuration unless indicated otherwise.

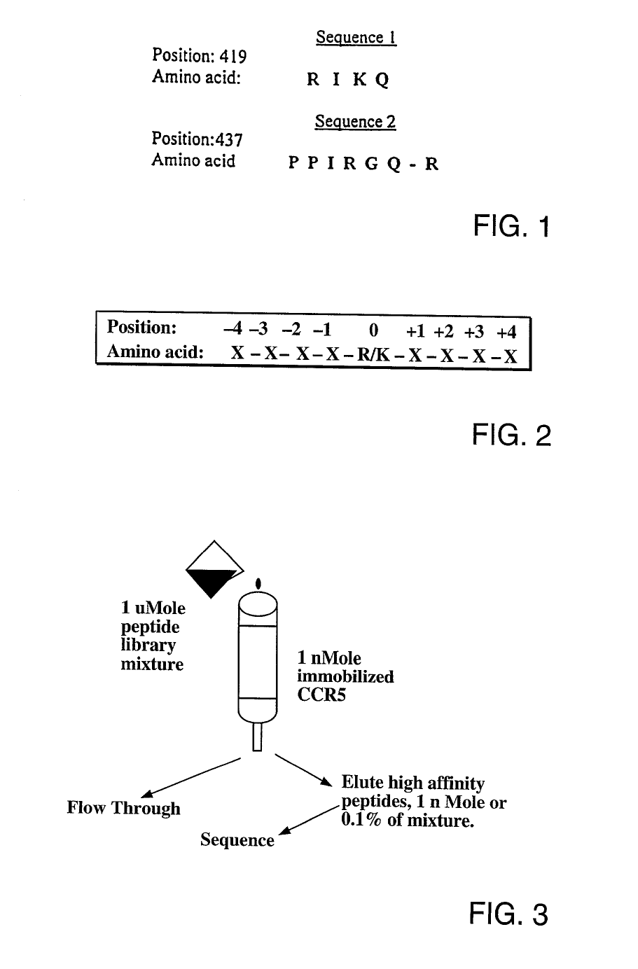

In general, amino acids from the residues of gp120 that are crucial for viral uptake may be used to specify fixed, or non-degenerate positions in the peptide libraries for use in the oriented peptide library method described below and in U.S. Pat. No. 5,532,167, the disclosure of which is incorporated by reference herein. See e.g., Rizzuto CD, et al., Science 280(5371):1949-53 (1998). See also FIG. 1.

Certain embodiments of the invention are described in the following examples, which are not meant to be limiting.

### EXAMPLES

### Example 1

### Preparation of Tagged CCR5; Screening of Linear Peptide Libraries

Various CCR5 vectors were prepared for the baculovirus expression system containing epitope tags using standard techniques known by those skilled in the art that allowed for easier purification of the receptor. Tags may be incorporated at the N- or C-terminus of the proteins. For CCR5, tags were incorporated at the C-terminus of the receptor to determine the character of the receptor's ligand-binding properties that are in the N-terminal or extracellular region of the molecule. The construction of a C-terminal 6xHis tagged and C-terminal FLAG construct are provided below as examples. Alternative tags may include, for example, GST, V5, Xpress, c-myc, HA, CBD, and MBP. These constructs were made by analogous procedures using standard techniques known by those skilled in the art.

The 6xHis tag enables a one-step purification using nickel chelation. The cDNA for CCR5 was obtained from Receptor Biology (Beltsville, Md.). To create a C-terminal tag, the CCR5 was digested out of the commercial vector, pcDNA3, and ligated into an E. coli vector, pET30a, with a C-terminal 6xHis tag. The newly created CCR5-6xHis was then excised and ligated into pBlueBac, a baculovirus transfer vector (Invitrogen, Carlsbad, Calif.). The construct was analyzed using both restriction digestion and sequencing, and then transfected into Sf9 insect cells (Pharmingen, San Diego, Calif.) for expression as typically done by those skilled in the art of protein expression.

A C-terminal bacterial FLAG construct was obtained from Sigma (pFLAG-CTC). A similar strategy using standard techniques was employed for the construction of this vector. The CCR5 was excised from pcDNA3, ligated with a similarly digested pFLAG-CTC plasmid, then excised with the C-terminal FLAG tag and ligated into the digested pBlueBac vector. The construct was analyzed using both restriction digestion and sequencing, and transfected into Sf9 or High Five (obtained from Invitrogen) insect cells for expression.

To express the CCR5 gene in Sf9 or High Five cells, the pBlueBac vector containing the CCR5 insert was cotransfected with Bac-N-Blue DNA using cationic liposome mediated transfection using standard techniques. The CCR5 was inserted into the baculovirus genome by homologous recombination. Cells were monitored from 24 hours post-transfection to 4-5 days. After about 72 hours, the transfection supernatant was assayed for recombinant plaques using a standard plaque assay. Cells which have the recombinant virus produced blue plaques when grown in the presence of X-gal (5-bromo-4-chloro-3-indoyl-β-D-galactoside). These plaques were purified and the isolate was verified by Polymerase Chain Reaction (“PCR”) for correctness of recombination using standard techniques. From this, a high-titer stock was generated and infection performed from this stock for expression work using standard techniques. Controls for transfection include cells only and transfer vector.

Sf9 or High Five cells were maintained both as adherent and suspension cultures using standard techniques known to those skilled in the art. The adherent cells were grown to confluence and passaged using the sloughing technique at a ratio of 1:5. Suspension cells were maintained in spinner flasks with 0.1% pluronic F-68 (to minimize shearing) for 2-3 months by sub-culturing to a density of 1×10⁶ cells/ml.

A time course after infection with recombinant virus was used to define optimal growth conditions for expression using standard techniques. Aliquots of cells from spinner flasks were taken for this time course, centrifuged at 800×g for 10 minutes at 4° C. and both supernatant and pellet assayed by SDS-PAGE/Western blot analysis. The CCR5 was expected to be in the membrane fraction (pellet). All viable systems were assayed in this fashion for levels of expression. Systems were assayed for activity using a standard binding assay on a membrane preparation using MIP-1α (Calbiochem, San Diego, Calif.) and [¹²⁵I]-MIP-1β (New England Nuclear, “NEN”, Boston, Mass.).

The membrane fraction was isolated by first pelleting the whole Sf9 cells (800×g for 10 minutes at 4° C.), then resuspending the pellet in a lysis buffer with homogenization. Typical lysis buffer is around neutral pH and contains a cocktail of protease inhibitors, both of which are standard techniques for those skilled in the art. Membranes were pelleted. Solubilization was also conducted using varying NaCl concentrations. Despite conventional thinking, the step of solubilization using low salt, for example, low calcium and magnesium concentrations substantially in the absence of NaCl provided unexpected optimal conditions for solubilization when compared for quantity and activity. Having 0.0 nM NaCl, although counter-intuitive, provided the best conditions when solubilizing and immobilizing candidates with the binding property of CCR5. The solubilization of the receptor by different detergents, such as, for example, β-dodecylmaltoside, n-octyl-glucoside, CHAPS, deoxycholate, NP-40, Triton X-100, Tween-20, digitonin, Zwittergents, CYMAL, lauroylsarcosine, etc., was compared for quantity and activity. A candidate for isolation was carried through for purification as described below.

After determining an appropriate detergent for solubilization and activity, such as, for example, NP-40, CCR5 was purified from the membrane fraction. The exact purification scheme will depend on the construct chosen, which is subject to activity and ease of solubilization. For purification of the 6xHis-tagged CCR5, the membrane fraction was loaded onto a Ni-NTA column (Qiagen, Valencia, Calif.) in the presence of detergent, washed extensively, and eluted with imidazole. Purification of the FLAG-tagged CCR5 was performed using the anti-FLAG M2 affinity matrix (Sigma, St. Louis, Mo.) in the presence of NP-40 at 0.3% concentration for solubilization and 0.1% concentration for immobilization and eluted with glycine. The purification was performed in the presence of NP-40. Activity of the purified receptor was assessed using a standard binding/displacement assay using MIP-1α and [¹²⁵I]-MIP-1β.

Peptides were assembled on Rink amide resin (NovaBiochem, substitution level 0/0.54 mmol/g) using an Applied Biosystems 433A synthesizer via 9-fluorenylmethyloxycarbonyl/tert.-butyl (“Fmoc”/“tBu”) based methods. tBu was used for the protection of side-chains of Asp, Glu, Ser, Thr, and Tyr, tert.-butyloxycarbonyl (“Boc”) for Lys and Trp, 2,2,4,6,7-pentamethyldihydrobenzofuran-5-sulfonyl (“Pbf”) for Arg, and triphenylmethyl (“trityl”, “Trt”) for Cys, His, Asn and Gln. The scale of the synthesis was 0.20 mmol. The resin was initially washed with N-methylpyrrolidinone (“NMP”) followed by a 1×3 minutes and 1×7.6 minutes treatment of piperidine:NMP (1:4) for N^{α}-Fmoc removal. All Fmoc-amino acids were coupled with N-[(1 H-benzotriazol-1-yl)(dimethylamino)methylene]-N-methylmethanaminium hexafluorophosphate N-oxide (“HBTU”) according to the manufacturer's protocol: (a) 1.0 mmol of derivatized amino acid was dissolved in 2.1 g of NMP; (b) 0.9 mmol of 0.5 M HBTU in N,N-dimethylformamide (“DMF”) was added to the amino acid cartridge and the solution was mixed for 6 minutes; (c) 1.0 mL of 2.0 M N,N-diisopropylethylamine (“DIEA”) in NMP was added to the cartridge; (d) the HBTU solution was transferred to the resin and reacted for 40 minutes at ambient temperature while mixing. The resin was filtered and rinsed six times with a total of 90 ml of NMP and the cycle was repeated. In the one pot method to construct the highly degenerate oriented peptide libraries, a batch of resin was allowed to react with mixtures of the combinatorial amino acids without any partitioning of the resin.

Adjusting the concentrations of the amino acids in the starting mixture controls the relative coupling rates, thereby ensuring equal incorporation of the amino acids in the library. The optimization of a mixture of natural Boc and Fmoc protected amino acids for the one pot synthesis has been previously described (see e.g., U.S. Pat. No. 5,225,533; Ivanetich, et al., Combinatorial Chemistry, vol 267, Academic Press, San Diego, Calif. USA, p 247-260 (1996); Buettner, et al., Innovations and Perspectives in Solid Phase Synthesis: Peptides, Proteins, and Nucleic Acids, Mayflower Worldwide Ltd., Birmingham, UK, p 169-174 (1994); Ostresh, et al., Biopolymers 34:1681-9 (1994); Songyang, et al., Methods in Mol Biol 87:87-98 (1998); and Herman, et al., Molecular Diversity 2:147-155 (1996). Cleavage reactions were performed by stirring the peptidyl-resin in trifluoroacetic acid (“TFA”):H₂O:anisole: triisopropylsilane (“iPr₃SiH”) (87.5:5:5:2.5, ˜6 mL) for 3 hours at 25° C. (see e.g., Herman et al., 1996). The filtrates were collected and the resin was further washed with TFA. Cold (−78° C.) diethyl ether was added to the combined extracts and the solution was cooled to −78° C. After removing the supernatant, the obtained precipitate was washed several times with cold ether, dissolved in glacial acetic acid and lyophilized.

For cyclic peptide libraries, Fmoc-Asp(OH)-ODmab (Dmab, 4-[N-(1-(4,4-dimethyl-2,6-dioxoxcyclohexylidene)-3-methylbutyl)amino]-benzyl) was side-chain anchored to Rink amide resin followed by chain elongation as described above. Following linear assembly, removal of the Dmab and Fmoc group was accomplished by treatments with hydrazine:DMF (1:49) for 7 minutes and piperidine:NMP (1:4) for 6×3 minutes, respectively. The resin was transferred to a syringe containing a polypropylene frit for manual cyclization. On-resin head-to-tail cyclization was performed using 7-azabenzotriazol-1-yloxy)-tris(pyrrolidino)phosphonium hexafluorophosphate (“PyAOP”):DIEA (1:2, 4 equiv) in a solution containing 1% Trition X in NMP:DMF:dichloromethane, methylene chloride, DCM) (1:1:1) for 2 hours at 55° C. The unreacted linear precursor was treated with Fmoc-Nva-OH/PyAOP/DIEA (“Nva”, “norvaline”)(1:1:2, 4 equiv) in DMF for 1×18 hours and 1×3 hours. Subsequent cleavage and side-chain deprotection as described above yielded a mixture containing a cyclic peptide library and the corresponding linear (uncyclized) sequences. The desired cyclic peptide library was purified to remove the linear contaminants by reversed-phase high performance liquid chromatography (“RP-HPLC”).

Peptides and peptide libraries were characterized by HPLC, MALDI-TOF MS (Louisiana State University), and Edman degradation. MALDI-TOF MS analysis is capable of detecting the presence of high-molecular weight impurities due to incomplete deprotection, deblocking, or re-alkylation. Edman degradation provides quantitative information about the amount of each amino acid in each degenerate position in a library.

The initial libraries synthesized had single, non-degenerate orienting amino acids (i.e., M-X-X-X-X-R-X-X-X-X-A, where X is a degenerate equimolar mixture of all amino acids except cysteine). Cyclic libraries (head-to-tail) were also prepared with single, non-degenerate orienting amino acids. Through the use of these initial libraries, the optimal residues at some degenerate positions become defined and secondary libraries were made fixing these positions. For example, the head to tail cyclized library cyclo(M-X-X-X-X-R-X-X-X-X-N) indicated that the −4 position (from the fixed R) should be lysine, the −2 position should be aspartate, the −1 position should be histidine, and the +3 position should be lysine so the secondary library was cyclo(M-K-X-D-H-R-X-X-K-N).

An oriented linear peptide library was applied to a column containing immobilized CCR5 and a small fraction of isolated high affinity peptides. A schematic diagram showing the peptide library screening using binding domains can be seen in FIG. 3. After washing, bound peptide library members are eluted from the column. Next, both the bound peptides and the entire library applied to the column were submitted individually to Edman degradation, to determine the distribution of amino acids as a function of position. Finally, the preferences of amino acids at the degenerate positions are determined. For example, if serine was 5% of the amino acids at position +1 in starting library but 15% of the amino acids in position +1 in the high affinity peptides, there would be a selection for serine at the +1 position. A preference value of 3 at that position would be obtained. Table 1 provides a selective review of the use of the peptide library method with binding domains.

TABLE 1
Use Of Oriented Linear Peptide Libraries To Determine Preferred
Amino Acids For Binding Domains
(residue used for orienting sequence is shown with underline)
(p = phospho-) -- “pX”

| Binding Domain | Preferred Peptide | Kd (nM) |
|---|---|---|
| PDZ | KKKKETDV | 42 |
| Src | EPQpYEEIPIYLK | 80 |
| 14-3-3 | RLSHpSLP | 55.7 |
| SH2 (src) | PYBEIY | 100 |
| SH3 | PXRPXR | |
| (amphiphysin) | | |
| SHC | NPXpY | |
| Lim | GPHydGPHydY/F | |

### Example 2

### Preparation of GST Tagged CCR5; Screening Using Same

Libraries were synthesized on an ABI 433A (Applied Biosystems, Forster City, Calif.) with 9-fluorenylmethoxycarbonyl (Fmoc) protecting groups using a Rink Amide MBHA resin (substitution: 0.54 mmol/gm). To obtain approximately equal coupling of amino acids for degenerate positions, the amounts of amino acids are adjusted empirically after considering literature values. See e.g., Ostresh J M, et al., Biopolymers 34(12):1681-9 (1994). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS (Louisiana State University) and by amino acid sequencing.

The initial library used a single, non-degenerate basic amino acid (i.e., M-A-X-X-X-X-R-X-X-X-X). However other libraries can be made with additional non-degenerate amino acids taken from sequence 1 or sequence 2 of the gp120 loop binding CCR5, see FIG. 1. Examples are X-X-R-I-X-Q-X-X based on sequence 1 or P-P-X-R-X-X-X-X based on sequence 2. The dash indicates that the residue is not involved. Secondary libraries were made fixing optimal residues found at some degenerate positions. Through the use of these initial libraries, the motifs become defined. For example, M-A-X-X-X-X-R-X-X-X-X indicated that the −4 position should be proline so the secondary library would be M-A-W-X-X-X-R-X-X-X-X.

In the case of the GST tagged CCR5, the receptor was exposed to the library, and separation of free and bound peptides was accomplished by pelleting the membranes by centrifugation. The GST tagged CCR5 purified receptor was incubated with a peptide library, about 1 μmole of peptide and about 1 nmole of binding sites. After incubation, receptor with bound peptide was separated from unbound peptides by centrifugation (receptorepeptide complex in the pellet, unbound peptide in the supernatant). Nonspecifically bound peptides were removed by exhaustive washing, and resuspension of the pellet in low pH (≦2.5) was used to remove the bound peptide. This peptide was sequenced to determine the consensus sequence.

When a FLAG-tagged CCR5 was used for peptide library work, the receptor was immobilized on an anti-FLAG M2 affinity matrix (St. Louis, Mo.). An additional purification approach used the CCR5-GST construct and immobilized glutathione (Pierce, Rockford, Ill.).

Both the bound peptide mixture and the starting peptide library were sequenced using standard techniques. The amounts of each amino acid, as a function of position, were determined. Preference values for each amino acid at each position were calculated by comparing the amounts of amino acids present in the starting library and bound fraction of peptides. These procedures were used to generate preferred sequences of peptides interacting with many binding domains and have been described in Table 1.

Also, secondary libraries were sequenced incorporating information from the initial library. For the first round of characterization, phage display technology is also used to identify preliminary binding motifs. The phage display method provides for the identification of motifs of natural amino acids. Phage display technology involves the insertion of DNA sequences into a gene coding for one of the phage coat proteins. The gene is inserted in a particular location so that the expressed protein insert can interact with other molecules. As a result, the encoded peptide or protein sequence will be presented on the surface of the phage and exposed for binding. By inserting degenerate nucleotides, each phage can express a different peptide sequence (“a phage library”). Incubation of this phage library with the immobilized receptor can be used to identify sequences which specifically bind to the receptor. Even weak signals can detected because they can be amplified by growing the isolated phage. Information derived from phage display is applicable to affinity purification methods using synthetic libraries containing novel amino acid analogs or cyclic peptides to select ligands that have enhanced pharmaceutical characteristics. The use of initial, secondary and tertiary libraries provided a complete definition of the specificity of the binding site.

Once preferred amino acids residues were identified using high preference values by CCR5 at the degenerate positions of the library, specific peptides were synthesized by methods as employed for library synthesis. Peptides were then purified by HPLC and compositions confirmed by MALDI-TOF MS.

Relative affinities were measured by modifying the radiolabel binding assay used in receptor purification. Therefore, the ability of these peptides to displace [¹²⁵I]-MIP-1β from purified CCR5 membranes was measured.

### Example 3

### Preparation and Analysis of Tagged CCR5; Screening Using Same

A. Cloning and Expression

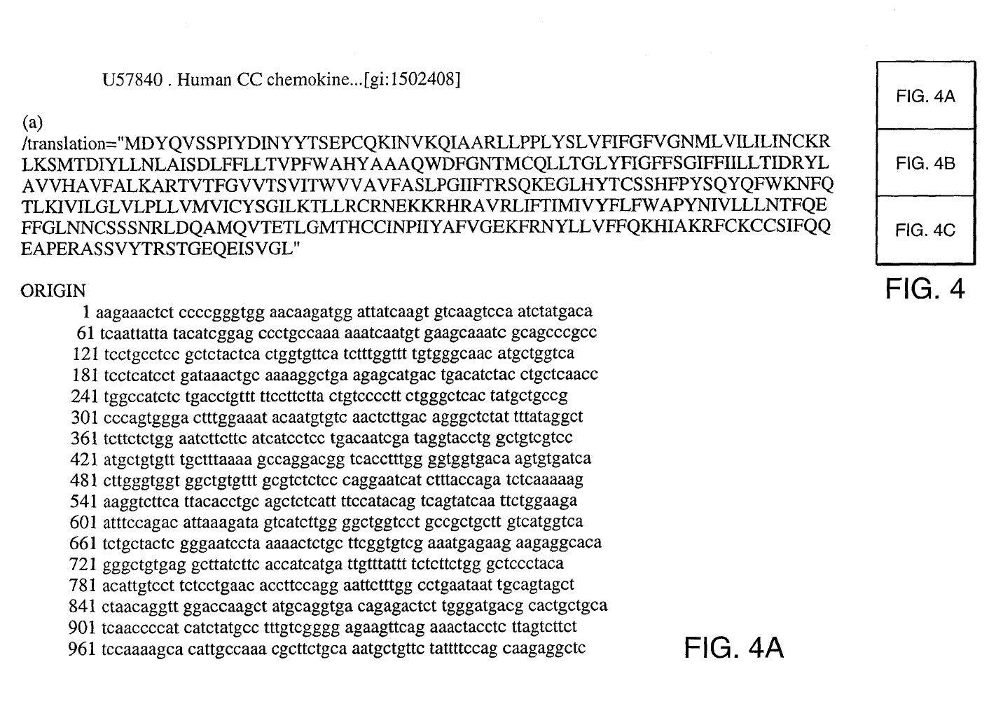

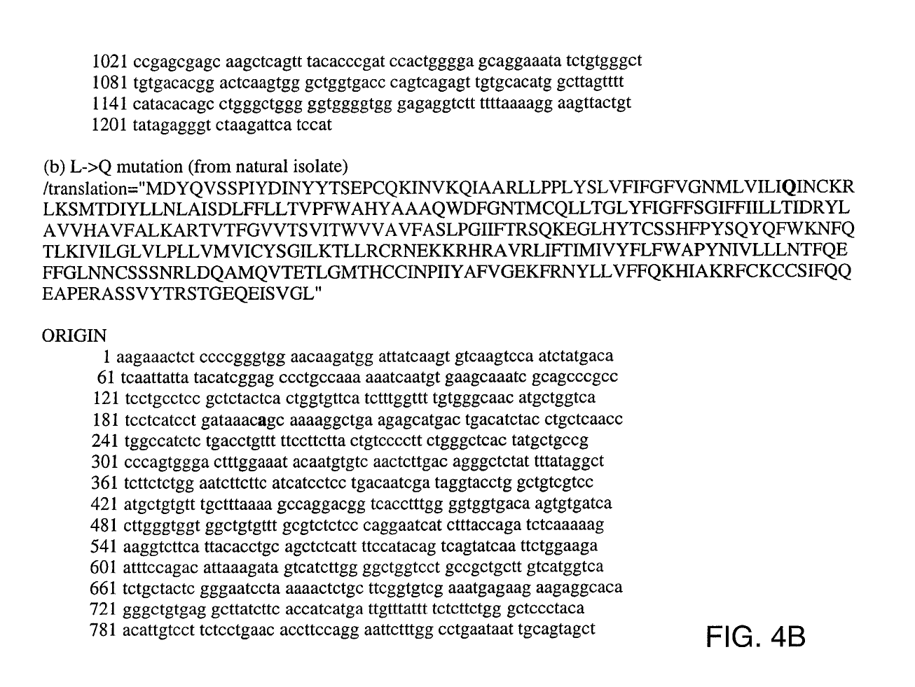

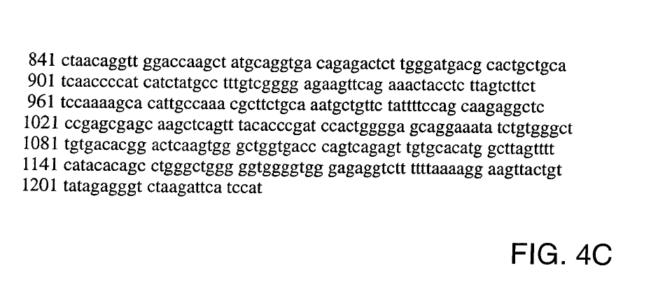

CCR5 cDNA (see sequence in FIG. 4) was obtained from Receptor Biology (Beltsville, Md.) in the vector pCDNA3. Tags were added to the C-terminus of the receptor for use in immobilizing them for affinity purification assays using standard techniques, as described herein. In addition, CCR5 cDNA correlating to GenBank Accession #U57840 may be used in the vector pCDNA3. The only difference between CCR5 cDNA from Receptor Biology and CCR5 cDNA correlating to GenBank Accession #U57840 is a point mutation at base 180 that changes Leucine to a Glutamine in the amino acid sequence. The following are specific examples from experiments using this tagging method with the CCR5 cDNA obtained from Receptor Biology:

### Construction of CCR5 with C-Terminal Histidine Tag (Insect Select Expression System)

The CCR5-HIS construct was derived from this system. PCR was performed using CCR5/pcDNA3 (from Receptor Biology) as the template and primers 5-Age His and 5-Spe His. The first primer introduced a unique Spe I site just before the initiator ATG of CCR5. The second primer mutated the stop codon of CCR5 into an Age I site in-frame with the histidine tag of pIZT/V5-His (Invitrogen, Carlsbad, Calif.). The PCR product was digested with Spe I and Age I, then ligated into similarly digested pIZT/V5-His. This construct is identified as CCR5-His-PIZT.

### Construction of CCR5 with C-Terminal Histidine Tag (Baculovirus Expression System)

CCR5-His-PIZT was digested with Hae II and Spe I, then filled in with Klenow fragment. The fragment containing CCR5-His was ligated into pBluebac 4.5 (Invitrogen,) that was previously digested with Nhe I and blunted with Klenow. The construct was checked for correctness of orientation. This is called CCR5-HIS. The construct was confirmed by restriction digestion and sequencing using standard techniques. This construct has been used for expression and has been determined to be expressed sufficiently and in active form for use in the affinity purification screening.

### Construction of CCR5 with C-Terminal FLAG Tag

PCR using standard techniques was performed using CCR5/pcDNA3 (from Receptor Biology) as the template and the primers 5-Xho pFLAG and 5-Sal pFLAG. The first primer engineered a unique Xho I site just before the initiator ATG of CCR5. The second primer mutated the stop codon of CCR5 into a Sal I restriction site (in-frame with the FLAG tag of pFLAG-CTC from Sigma). The PCR product was digested with Xho I and Sal I and ligated into similarly digested pFLAG-CTC (a bacterial expression vector). This construct is called CCR5-FLAG-CTC. CCR5-FLAG was then digested with Xho I and Sca I, and filled in with Klenow fragment. The fragment containing CCR5-FLAG-CTC was ligated into pBluebac 4.5 that was first digested with Nhe I then blunted with Klenow. This final construct is identified as CCR5-FLAG. The construct was confirmed by restriction digestion and sequencing using standard techniques. This construct has been used for expression and has been determined to be expressed sufficiently and in active form for use in affinity purification screening.

### Construction of CCR5 with C-Terminal GST Tag

PCR was performed using CCR5/pcDNA3 (from Receptor Biology) as the template and primers GST-BamHI and GST-Nde1. The first primer mutated the stop codon of CCR5 into a BamH I restriction site (in-frame with the FLAG/GST tag of pESP-3). The second primer introduced a unique Nde I site at the initiator codon of CCR5. The PCR product was digested with BamH I and Nde I and ligated into similarly digested pESP-3 (a yeast expression vector.) This construct is called pCP8. pCP8 was then digested with Nde I and Sma I, and filled in with Klenow fragment. The fragment containing CCR5-GST was ligated into pBluebac 4.5 that was first digested with Bgl II then blunted with Klenow. This final construct is identified as pCP10. When describing the protein, the construct is identified as CCR5-GST. The construct was confirmed by restriction digestion and sequencing using standard techniques. This construct has been used for expression and has been determined to be expressed sufficiently and in active form for use in affinity purification screening.

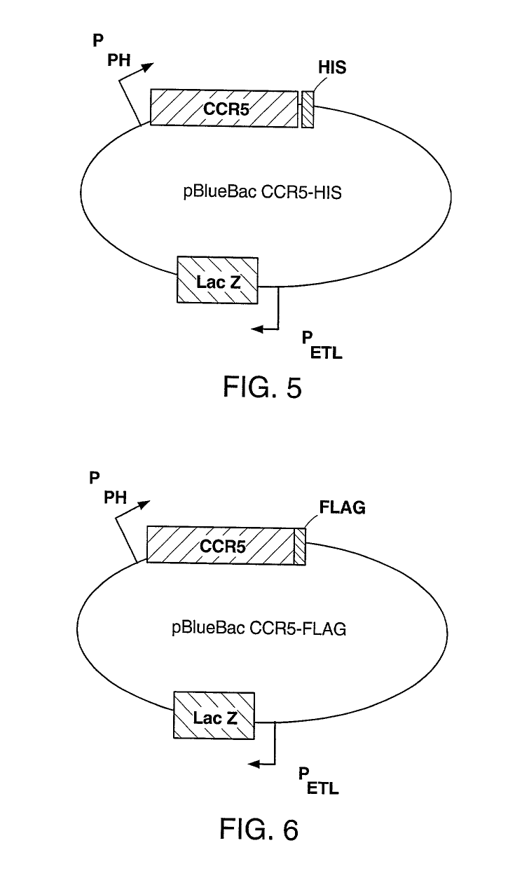

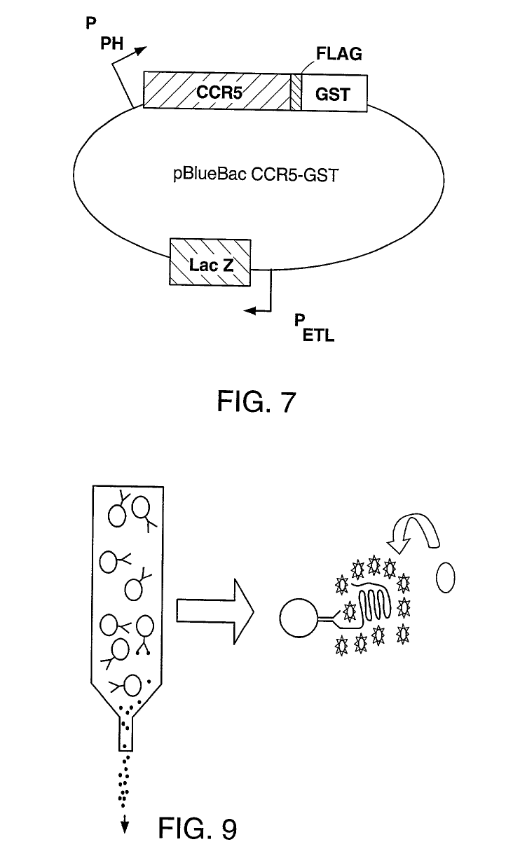

Plasmid maps for these vectors can be found in FIGS. 5-7.

The vectors for the three new constructs (for CCR5-FLAG, CCR5-GST, and CCR5-HIS) were used to co-transfect Sf9 cells for the production of a viral stock of each. These viral stocks were purified using a standard plaque assay and then used in experiments to infect for the optimization of expression of CCR5 with its various C-terminal tags. High Five cells (Invitrogen) were also transfected with these CCR5 tagged constructs and tested for expression of CCR5. All constructs were determined to express the appropriately tagged receptor. Expression levels after 72 hours were as much as 5 times greater in High Five cells than those for Sf9 cells. All of the above described experiments were done using standard techniques known to those skilled in the art.

A fourth construct for the expression of CCR5 was made from the starting vector pBlueBac 4.5 (Invitrogen) to remove the thrombin and enterokinase cleavage sites in the previously described vectors. The GST tag was added into the multiple cloning site by using PCR to generate the GST tag, then ligating into the digested vector (SmaI/EcoRI) using standard procedures known to those skilled in the art. Next, the vector was made compatible with the Gateway technology from Lifetech for ease of manipulation. This was done by ligating into the SmaI site the cassette containing the recombination sites required for this technology (from Lifetech). CCR5 was amplified using PCR with primers to extend the gene to contain the attachment sites for recombination. Then, the PCR product was incorporated into the baculovirus vector using BP clonase (the enzyme required for homologous recombination) to make a vector for baculovirus expression containing CCR5 with a C-terminal GST tag without the enterokinase or thrombin cleavage sites. This vector was cotransfected into Sf9 cells for preparation of the virus stock necessary for expression. The virus was plaque purified, and a PCR and sequence checked clone was used for expression of CCR5. A time course with this construct showed that less proteolysis of the protein was observed and less time was necessary to obtain maximal expression of the receptor.

B. Activity

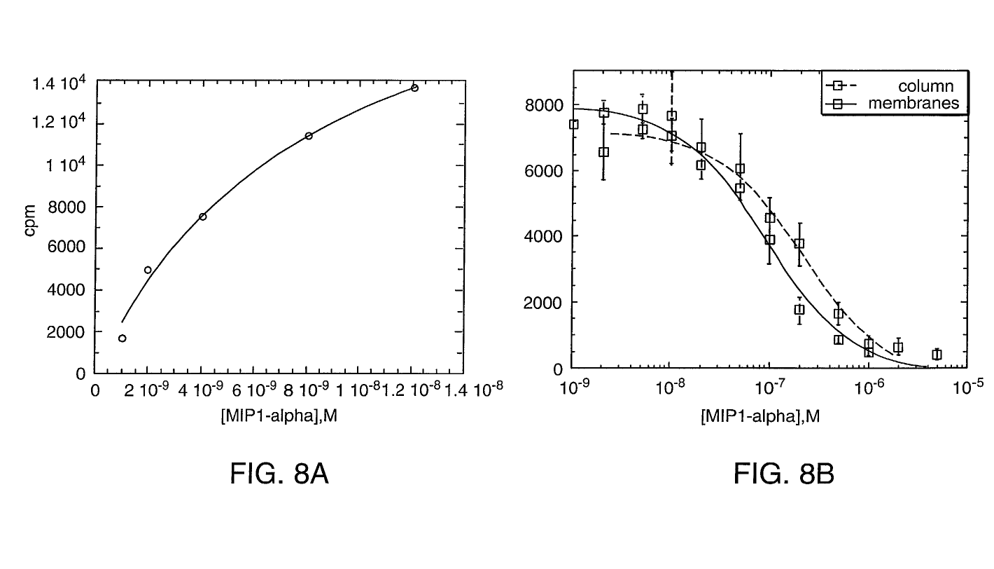

Each of the tagged CCR5 genes (CCR5-GST, CCR5-FLAG, and CCR5-HIS) were expressed in Sf9 and High Five cells, as described in Example 1. Whole cells from Sf9 and High Five cell lines were lysed using hypotonic buffers (10 mM Tris, pH 7.4, 5 mM EDTA), and membrane preparations were made by homogenization and centrifugation using standard techniques known to those skilled in the art. Membrane preparations for CCR5-GST, CCR5-FLAG, and CCR5-HIS were assayed using a standard radioligand binding assay respectively. Binding assays were performed with 5 μg of membranes in 50 mM Hepes, pH 7.5, 1% BSA. Incubation was for 1 hour at 27° C. Incubation was done in a 96-well PCR plate and transferred to a 96-well filter-punch plate (Millipore) for washing (with buffer containing 250 mM NaCl to reduce nonspecific binding) and counting. All samples were assayed in triplicate. FIG. 8 generally shows charts exemplifying the characterization of CCR5 receptor binding. Specifically, the K_{d} for binding was measured using saturation experiments by adding increasing amounts of [¹²⁵I]-MIP-1β to CCR5 as seen in FIG. 8a. Displacement of this radioligand (1 nM [¹²⁵I]-MIP-1β) to CCR5 for IC₅₀ determination was measured using MIP-1α both in membrane preparations and immobilized on the column as seen in FIG. 8b. Binding of MIP-1β was both saturable and reversible.

Uninfected cells were used as a control for this experiment. The activity of the membrane preparations was comparable to that obtained by Receptor Biology (K_{d}<1 nM for MIP1-β binding) at least having 20% active protein.

C. Solubilization

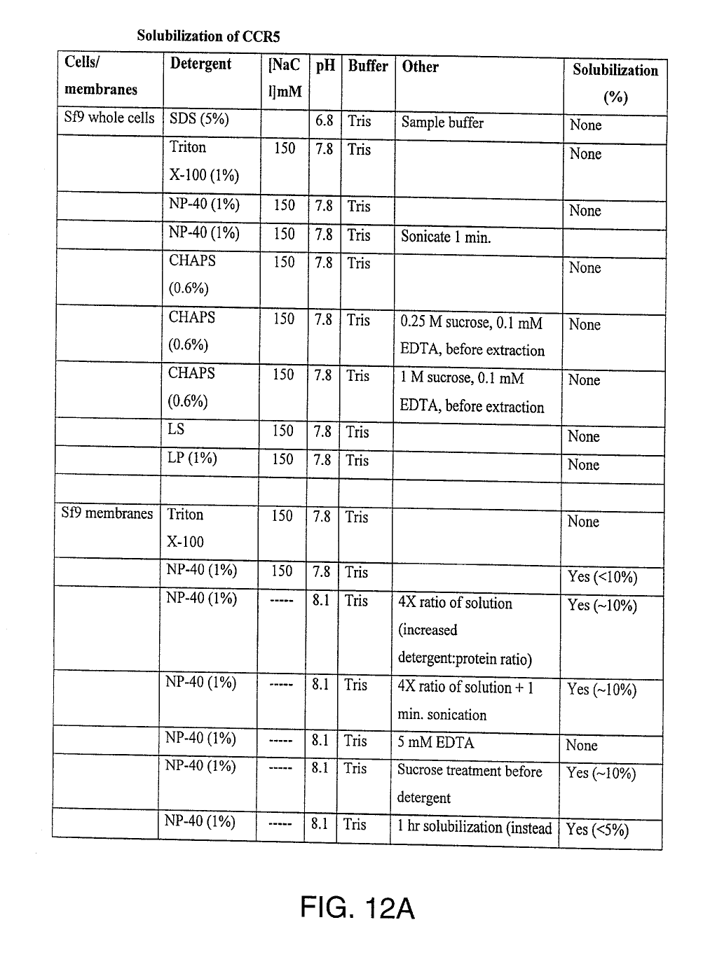

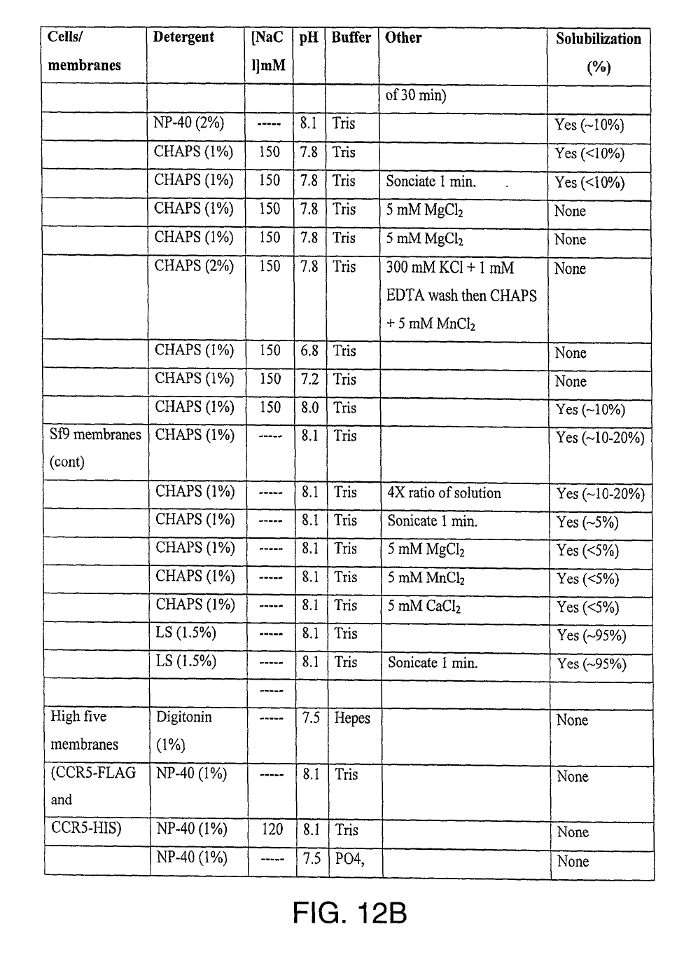

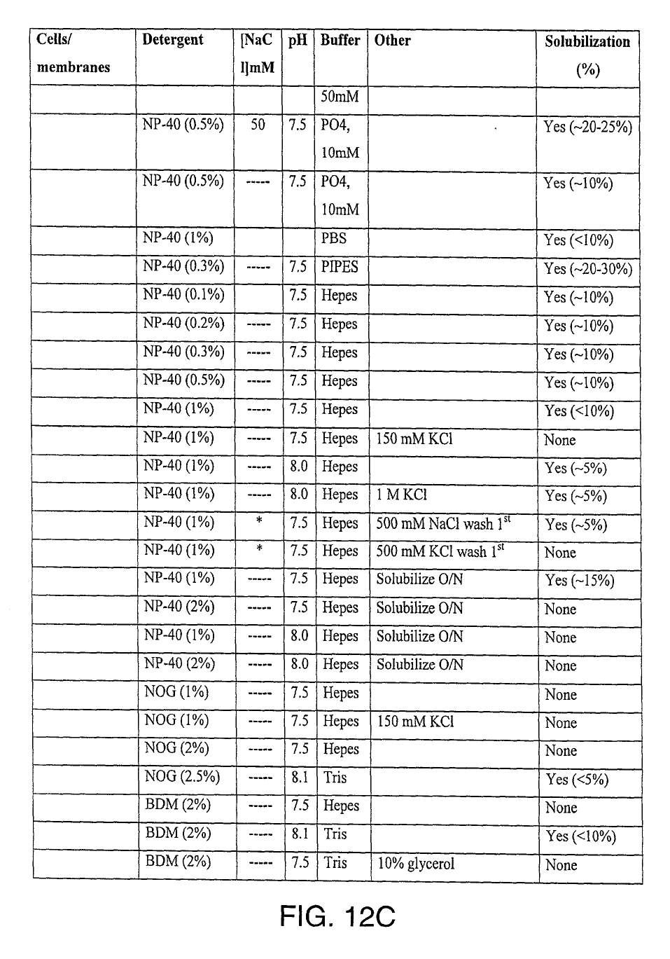

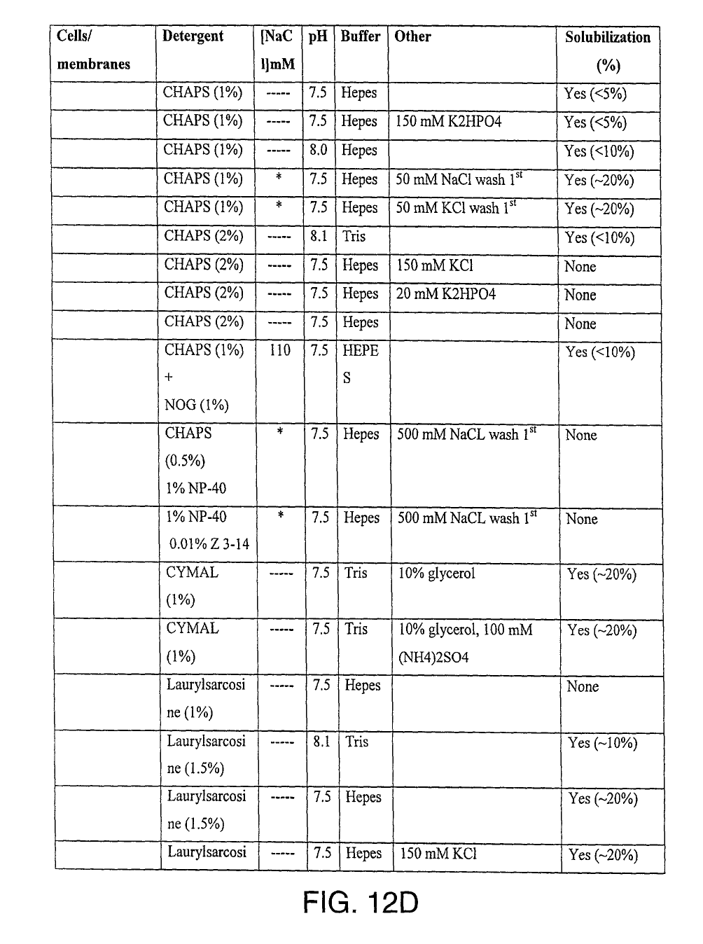

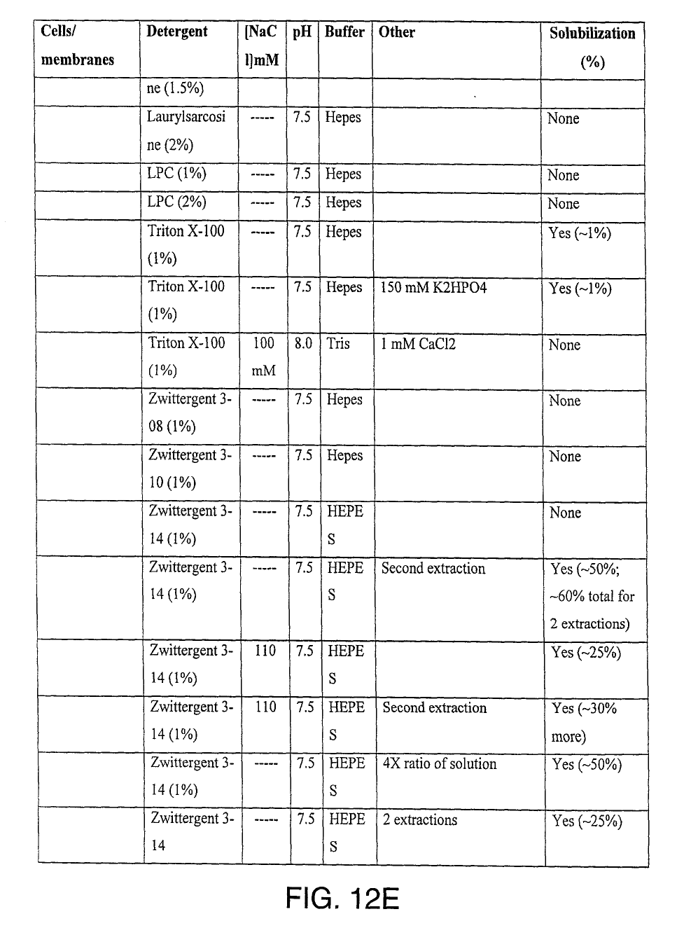

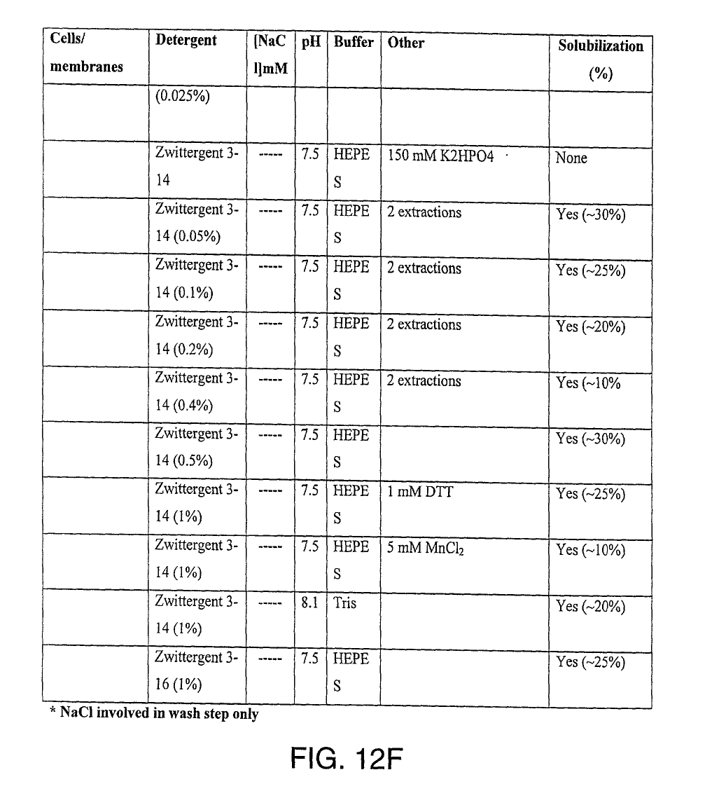

Both lysed whole cells and membrane preparations have been used for solubilization. Solubilization of the tagged versions of CCR5 (CCR5-FLAG, CCR5-GST, and CCR5-HIS) have been performed using many different combinations of detergents (NP-40, Triton X-100, β-D-maltoside, n-octylglucoside, CYMAL, Zwittergents, Tween-20, lysophosphatidyl choline, CHAPS, etc.), salts (NaCl, CaCl₂, MgCl₂, MnCl₂, KCl, etc.), buffers (Tris, Hepes, Hepps, Pipes, Mes, Mops, acetate, phosphate, imidazole, etc.), and various pH's (range 6.8-8.2). Conditions for optimal solubilization were found using Zwittergent 3-14 and low salt, e.g. low magnesium and calcium, but no NaCL (0.0 nM NaCl) at pH 8.1. In a preferred embodiment, at least 20% of the solubilized, immobilized protein is active. In highly preferred embodiments, at least 30%, 40%, 50% and 75% of the solubilized, immobilized protein is active. FIG. 12 demonstrates the solubilization of CCR5 with some exemplary conditions used for these experiments and their respective results.

D. Immobilization

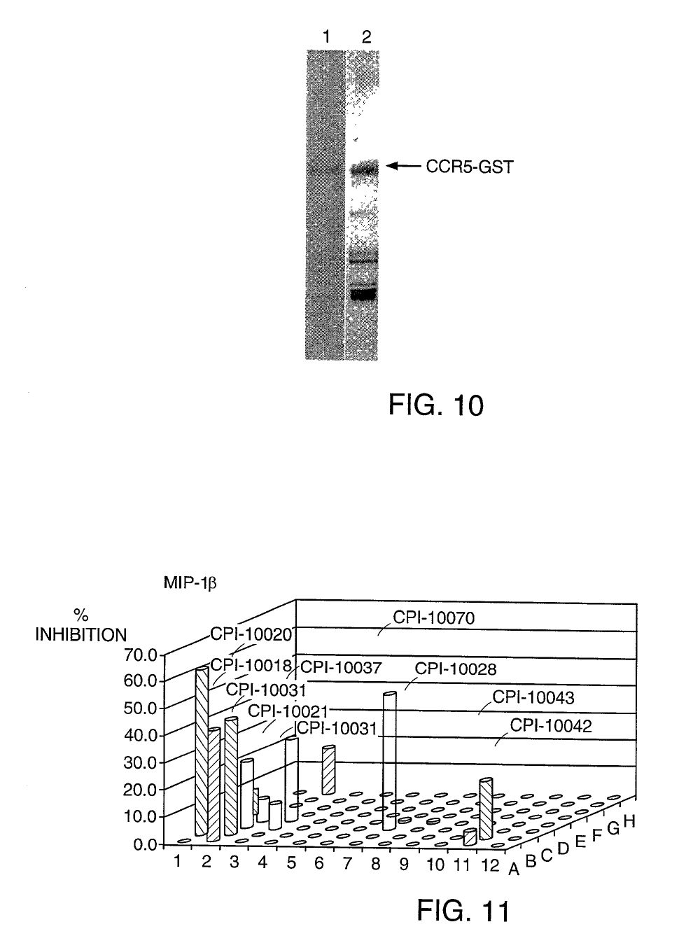

After solubilization, both CCR5-GST and CCR5-FLAG were immobilized onto affinity columns for purification and for use as active proteins for screening of peptide libraries. A schematic diagram showing the immobilization of GPCRs for affinity purification from libraries is shown in FIG. 9. CCR5-GST was bound and immobilized onto glutathione-agarose (Pierce) and glutathione-sepharose (Amersham Pharmacia Biotech) and CCR5-FLAG was immobilized onto a specific antibody column that recognizes the FLAG epitope (M2 column, Sigma). The immobilization of the functional protein was accomplished by first solubilizing the receptor in 0.3% NP-40, 10 mM Hepes (or Pipes), pH 7.5, then binding it to the glutathione-sepharose resin in 0.1% NP-40, 10 mM Hepes (or Pipes), pH 7.5, 3 mM CaCl₂, 15 mM MgCl₂. The activity of the immobilized receptor was determined by incubation for 1 hour at 4° C. with the radiolabeled MIP-1β (as with the membrane assay above) and competition with cold MIP-1α. Uninfected cells have been used as controls for this activity, as well as the column alone. These experiments demonstrated the ability to immobilize microgram quantities of the receptor in pure form (sufficient for affinity purification screening) onto resin in active form (see FIG. 8a ). FIG. 10 shows the eluant analyzed by SDS-PAGE and western blot for the purified receptor (coomassie stained) in Lane 1 and the α-GST antibody developed western blot of the solubilized CCR5-GST. Purification of the CCR5-GST was done using glutathione-sepharose. CCR5-GST was bound to glutathione-sepharose after solubilization with 0.3% NP-40. NP-40 was diluted to 0.1% before binding by rotation at 4° C. for 2 hours with the resin. After washing, still in the presence of detergent, CCR5-GST was eluted using 10 mM glutathione.

E. Peptide Library Synthesis

Libraries were synthesized on an ABI 433A (Applied Biosystems, Forster City, Calif.) with 9-fluorenylmethoxycarbonyl (Fmoc) protecting groups using a Rink Amide MBHA resin (substitution: 0.54 mmol/gm). When a mixture of amino acids was to be used for degenerate positions, the approximately equal coupling of amino acids was obtained by adjusting the amounts of amino acids empirically after considering literature values. See. e.g., Ivanetich et. al., Combinatorial Chemistry, vol 267, Academic Press, San Diego, Calif. USA, p 247-260 (1996). The coupling reagent was HBTU/HOBT/DIEA, 1 equivalent per equivalent of peptide. Cleavage was effected by a cocktail (82% TFA, 5% phenol, 5% thioanisol, 2.5% 1,2-ethanedithiol, 5% water). Peptides were precipitated from methyl tertiary butyl ether. Libraries were characterized by MALDI-TOF MS (Louisiana State University) and by amino acid sequencing.

The initial libraries used a single, non-degenerate basic amino acid (i.e., M-X-X-X-X-W-X-X-X-X-A-K-K-K). Through the use of these initial libraries, the optimal residues at some or all degenerate positions became defined. Secondary libraries were made if all of the positions were not defined, thereby fixing the defined positions.

F. Screening of Peptide Libraries Using Immobilized CCR5

With active, large quantities of protein (1 nmol) immobilized to the specific resin (for example, CCR5-GST to glutathione-sepharose or CCR5-FLAG to M2 antibody-agarose), screening of billions of compound can take place by incubating them together and allowing the natural preferences and binding affinities to purify the ligands which are preferred by CCR5. These experiments have been performed using the linear library 4P4(+) and 18 additional libraries. Additional experiments may be performed with other libraries.

To identify which peptide libraries to screen, a membrane binding assay was developed to use with the peptide libraries. Each peptide library (100 μM final concentration, average molecular weight) was incubated with the receptor (in membranes obtained as described herein) and the ability of the peptides in the library to inhibit [¹²⁵I]-MIP-1β binding was determined. Percent inhibition was calculated. Peptide libraries with the highest percent inhibition were assayed first. Specifically, as shown in FIG. 11, binding inhibition assays were performed using 5 μg membranes, 0.5 nM [¹²⁵I]-MIP-1β and a 100 μM aliquot of each peptide library. Results are shown in FIG. 11 as % inhibition. The best inhibition is observed with CPI-10070 (M-A-X-X-X-X-R-X-X-X-X-A) and CPI-10020 (M-X-X-X-R-X-X-X). The libraries include both linear and cyclic peptide libraries with various fixed orienting residues.

Approximately 500 mL of 1×10⁶ cell/mL of High Five cells expressing CCR5-GST were used per affinity purification. Immobilized CCR5-GST (as described herein) was incubated with 1 mg of the peptide library for 20 minutes at room temperature. Unbound peptides were removed by washing. Bound peptides were eluted with 30% acetic acid. Eluted peptide was filtered using a Centricon-10 to remove any protein that might have co-eluted with acetic acid. The filtrate was dried under vacuum, dissolved in water and subjected to peptide sequencing. Table 2 summarizes the results of the affinity purifications performed with CCR5 and peptide libraries. From primary screenings, secondary focused or orienting libraries were synthesized.

TABLE 2
Summary of results from CCR5 affinity purifications using peptide
libraries.

| | Bound | Control | |
|---|---|---|---|
| Library | (pmol) | (pmol) | Results |
| CPI-10020 | 101 | 44 | Identified sequence and focused library |
| | | | (cyclo(M-K-X-D-H-R-X-X-X-N)) |
| CPI-10101 | 876 | 743 | High nonspecific binding; identified focused library |
| | | | (cyclo(M-X-X-S-X-X-R-W-X-T-X-N)) |
| CPI-10070 | 1579 | 893 | High background but identified sequence |
| | | | (M-W-N-W-S-R-D-W-H-V-A) |
| CPI-10045 | 110 | 13 | One identified preference |

TABLE 3
Other Exemplary Libraries

| Library | Sequence |
|---|---|
| CPI-10018 | Cyclo(Met-[Xxx]3-Arg-[Xxx]2-Asn) |
| CPI-10021 | Cyclo(Met-[Xxx]₄-Lys-[Xxx]₃-Asn) |
| CPI-10028 | Cyclo(Met-[Xxx]₅-Tyr-[Xxx]₄-Asn) |
| CPI-10031 | Cyclo(Met-[Xxx]₄-Arg-[Xxx]₄-Asn)-Ala-[Lys]₃-NH₂ |
| CPI-10037 | Cyclo([Xxx]₁₁-Asn) |
| CPI-10042 | H-[Xxx]₃-Thr-Pro-Gly-Tyr-Trp-[Xxx]₃NH₂ |
| CPI-10043 | H-Phe-Glu-Trp-[Xxx]₅-Gln-Pro-Tyr-NH₂ |
| CPI-10064 | H-Met-Ala-[Xxx]₄-Trp-[Xxx]₄-Ala-[Lys]₃-NH₂ |

G. Phage Display

As an alternative or additional method useful in screening, immobilized functional CCR5 was used to isolate phage that bind to CCR5 using standard techniques known to those skilled in the art. Subtraction of the background from the glutathione-sepharose beads, BSA, and MIP1β used in the assay was performed by incubation of the phage library with the mixture of these components. After incubating subtracted phage libraries (i.e., NEB, PhD C7C) with the receptor, bound phage were eluted both with the natural CCR5 ligand (MIP1β) and with glycine, pH 2.2. PhD C7C is a particular phage library with 7 random amino acids between disulfides, and may be obtained from New England Biolabs. Multiple rounds of screening were performed. Both conditions have provided specific sequences which bind to CCR5 and may inhibit ligand binding.

TABLE 4
Sequences Identified Through Phage Display for CCR5 Binding

| | |
|---|---|
| 1 | SPAYPYSAPRTF |
| 2 | SPADFYSHPALH |
| 3 | TTAKHYFHPARH |
| 4 | VLNDLQTHPRLA |
| 5 | VPSPVAHPPFLI |
| 6 | SVRLITTAPHAP |
| 7 | SLIDFYNRQAFW |
| 8 | WSFDTPAFRSMH |
| 9 | TSPYFQSVSWGH |
| 10 | CRHSYVSSWC |
| 11 | CLTYGVSGDC |
| 12 | CHLSWDVPYC |
| 13 | LPIQCSLMFC |
| 14 | EARECGSGGC |
| 15 | NLWLCDGIFC |
| 16 | WSAGGDWR |
| 17 | EWAWVLDA |
| 18 | YGGTIIWW |
| 19 | SGSGFWLL |

### Example 4

### Prevention or Treatment of HIV Infection or AIDS

Presently, certain complications, however, are encountered during the production, formulation and use of therapeutic peptides, peptidomimetic, or small molecule antagonists or agonists of CCR5 binding used for the prevention and treatment of AIDS and HIV infection. Biologically appropriate antagonists that minimize the cost and technical difficulty of commercial production of therapeutic binding compounds of CCR5 are further contemplated by the present invention. In addition, biologically appropriate antagonists or agonists of CCR5 binding that do not confer an immunilogical response to the antagonist or agonist such that it interferes with the effectiveness thereof are contemplated by the invention. Moreover, appropriate formulations that confer commercially reasonable shelf life of the produced antagonist or agonist of CCR5 binding, without significant loss of biological efficacy are contemplated in the present invention. Furthermore, useful dosages for administration to an individual are contemplated in the present invention appropriate for the prevention and treatment of AIDS and HIV infection.

The identification of appropriate candidates that, alone or admixed with other suitable molecules, that are competent in inhibiting CCR5 binding are contemplated by the invention. Such candidates further contemplate the production of commercially significant quantities of the aforementioned identified candidates that are biologically appropriate for the prevention and treatment of AIDS and HIV infection. Moreover, the invention provides for the production of therapeutic grade commercially significant quantities of CCR5 binding antagonists, agonists or derivatives in which any undesirable properties of the initially identified analog, such as in vivo toxicity or a tendency to degrade upon storage, are mitigated.

Methods of preventing and treating AIDS and HIV infection also, after the identification and design of a peptide, peptidomimetic, or small molecule antagonist of CCR5 binding activity, comprise the step of administering a composition comprising such a compound capable of inhibiting CCR5 binding as described herein. Administration may be by any compatible route. Thus, as appropriate, administration may include oral or parenteral, including intravenous and intraperitoneal routes of administration. A particularly preferred method is by controlled-release injection of a suitable formulation. In addition, administration may be by periodic injections of a bolus of a composition, or may be made more continuous by intravenous or intraperitoneal administration from a reservoir that is external (e.g., an intravenous bag) or internal (e.g., a bioerodable implant).

Therapeutic compositions contemplated by the present invention may be provided to an individual by any suitable means, directly (e.g., locally, as by injection, implantation or topical administration to a tissue locus) or systemically (e.g, parenterally or orally). Where the composition is to be provided parenterally, such as by intravenous, subcutaneous, intramolecular, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition may comprise part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance.

Useful solutions for parenteral administration may be prepared by any of the methods well known in the pharmaceutical art, described, for example, in REMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. The concept of a controlled release injectable formulation for peptide drugs is well-accepted and offers several advantages. First, for example, bioavailabilities are high. Second, treatment regimens can consist of once per month or per three months (like Abbott's Leupron®), or once per year (e.g. Alza's Viadur®). Third, controlled release injectable formulations substantially reduces the doses that can be used (the Leupron injection dose is 1 mg/day but the 90 day formulation uses is 11.25 mg total). Also, increased efficacy can be achieved if the therapeutic is present continuously to prevent infectivity. This consideration is particularly important in view of the need to approach a cure for this disease by preventing the reformation of slow-to-clear deposits of infection such as the memory T cell compartment. See e.g., Lee, V., ed. Peptide and Protein Drug Delivery. Marcel Dekker, Inc., NY (1991).

Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or cutric acid for vaginal administration.

Additional aspects and embodiments of the invention are apparent to the skilled artisan.

We claim:

1. A method of identifying a binding compound for CC chemokine receptor 5 comprising the steps of:
a) providing a library of two or more molecules;
b) providing a molecule having a binding property corresponding to CC chemokine receptor 5, wherein said molecule is attached to a support;
c) binding a molecule from said library of two or more molecules to said molecule having a binding property corresponding to CC chemokine receptor 5 attached to said support;
d) separating said bound molecule from said molecule attached to said support; and
e) identifying said bound molecule as a binding compound for CC chemokine receptor 5.

2. The method of claim 1, wherein said library of two or more molecules is selected from the group consisting of linear peptides, cyclic peptides, natural amino acids, unnatural amino acids, peptidomimetic compounds and small molecule compounds.

3. The method of claim 1, wherein said molecule having a binding property corresponding to CC chemokine receptor 5 is a partially purified CC chemokine receptor.

4. The method of claim 1, wherein at least one of said two or more molecules is selected from a group consisting of a peptide, a peptidomimetic or small molecule that can substitute for a protein capable of binding to receptors, enzymes or other proteins.

5. The method of claim 1, further comprising the step of solubilizing said molecule having a binding property corresponding to CC chemokine receptor 5 substantially in the absence of sodium chloride.

6. The method of claim 1, further comprising the step of solubilizing said molecule having a binding property corresponding to CC chemokine receptor 5 using a buffer having a low salt concentration.

7. The method of claim 1, wherein at least one of said two or more molecules comprises a molecule having an antagonistic effect on CC chemokine receptor 5 binding activity.

8. The method of claim 1, wherein said library comprises a phage library.

9. The method of claim 1, wherein said steps a, b, c, and d are repeated at least once prior to said step e.

10. The method of claim 1, wherein said molecule having a binding property corresponding to CC chemokine receptor 5 comprises a CC chemokine receptor 5 molecule and a tag selected from the group consisting of GST, FLAG, 6xHis, C-MYC, MBP, V5, Xpress, CBP, and HA).

11. A binding compound for CC chemokine receptor 5 identified according to the method of claim 1.

12. A method of preventing HIV infection in a patient, the method comprising administering to said patient a therapeutic composition comprising the compound of claim 9 in a physiological carrier.

13. A method of treating or preventing AIDS in a patient, the method comprising administering to said patient a therapeutic composition comprising the compound of claim 9 in a physiological carrier.

14. A method of treating or preventing AIDS in a patient, the method comprising administering to said patient a therapeutic composition comprising the compound of claim 9 in a controlled release injectable formulation.

15. A computer-aided method for identifying relative binding affinity of a test molecule to CC chemokine receptor 5, comprising the steps of:
a) entering input data characterizing CC chemokine receptor 5 into a computer program;
b) entering input data characterizing at least one test peptide-like molecule, each of known sequence but unknown binding affinity;
c) analyzing each applied test peptide-like molecule using the computer program to generate a prediction of a relative binding affinity for each test peptide-like molecule, and outputting such prediction.

16. A method for determining an amino acid sequence motif for an interaction site of a binding compound for CC chemokine receptor 5, comprising the steps of:
a) contacting a peptide library with a molecule having a binding property corresponding to CC chemokine receptor 5 under conditions which allow for interaction between said molecule having a binding property corresponding to CC chemokine receptor 5 and said peptide library;
b) allowing said molecule having a binding property corresponding to CC chemokine receptor 5 to interact with said peptide library such that a complex is formed between said molecule having a binding property corresponding to CC chemokine receptor 5 and a subpopulation of library members capable of interacting with said molecule having a binding property corresponding to CC chemokine receptor 5;
c) separating said subpopulation of library members capable of interacting with said molecule having a binding property corresponding to CC chemokine receptor 5 from library members that are incapable of interacting with said molecule having a binding property corresponding to CC chemokine receptor 5;
d) linearizing said subpopulation of library members capable of interacting with said molecule having a binding property corresponding to CC chemokine receptor 5;
e) determining a relative abundance of different amino acid residues at each degenerate position within a mixture of linearized library members; and
f) determining an amino acid sequence motif for an interaction site of said molecule having a binding property corresponding to CC chemokine receptor 5, based upon said relative abundance of different amino acid residues at each degenerate position within the mixture of linearized library members.

17. An amino acid sequence motif for a binding compound for CC chemokine receptor 5 identified according to the method of claim 16.

18. An amino acid sequence motif identified according to the method of claim 16 having a sequence selected from the group consisting of M-A-R-S-L-I-W-R-P-A-K-A-K-K-A, K-K-K-A-R-S-L-I-W-R-P-A-K-A-K-K-K, and cyclo(M-Y-A-T-R-W-K-N).

19. A binding compound having the amino acid sequence motif for CC chemokine receptor 5 determined by the method of claim 16.

20. A binding compound identified according to the method of claim 8 having a sequence selected from the group consisting of S-P-A-Y-P-Y-S-A-P-R-T-F, S-P-A-D-F-Y-S-H-P-A-L-H, T-T-A-K-H-Y-F-H-P-A-R-H, V-L-N-D-L-Q-T-H-P-R-L-A, V-P-S-P-V-A-H-P-P-F-L-I, S-V-R-L-I-T-T-A-P-H-A-P, S-L-I-D-F-Y-N-R-Q-A-F-W, W-S-F-D-T-P-A-F-R-S-M-H, T-S-P-Y-F-Q-S-V-S-W-G-H, C-R-H-S-Y-V-S-S-W-C, C-L-T-Y-G-V-S-G-D-C, C-H-L-S-W-D-V-P-Y-C, L-P-I-Q-C-S-L-M-F-C, E-A-R-E-C-G-S-G-G-C, N-L-W-L-C-D-G-I-F-C, W-S-A-G-G-D-W-R, E-W-A-W-V-L-D-A, Y-G-G-T-I-I-W-W, and S-G-S-G-F-W-L-L.

21. The method of claim 16, wherein at least one member of said peptide library comprises at least one unnatural amino acid.

22. The method of claim 16, wherein said molecule having a binding property corresponding to CC chemokine receptor 5 is selected from the group consisting of linear peptides, cyclic peptides, natural amino acids, unnatural amino acids, peptidomimetic compounds and small molecule compounds.

23. The method of claim 16, wherein said peptide library comprises at least one molecule selected from the group consisting of linear peptides, cyclic peptides, natural amino acids, unnatural amino acids, peptidomimetic compounds and small molecule compounds.

24. The method of claim 16, wherein said peptide library is selected from the group consisting of CPI-10018, CPI-10020, CPI-10021, CPI-10028, CPI-10031, CPI-10037, CPI-10042, CPI-10043, CPI-10045, CPI-10064, CPI-10070 and CPI-10101.

25. The method of claim 16, wherein said peptide library is selected from the group consisting of M-A-X-X-X-X-R-X-X-X-X-A, M-X-X-X-R-X-X-X, M-X-X-X-X-R-X-X-X-X-A, M-A-X-X-X-X-R-X-X-X-X, X-X-R-I-X-Q-X-X, P-P-X-R-X-X-X-X, M-A-W-X-X-X-R-X-X-X-X, M-X-X-X-X-W-X-X-X-X-A-K-K-K, M-W-N-W-S-R-D-W-H-V-A, cyclo(M-X-X-X-X-R-X-X-X-X-N), cyclo(M-K-X-D-H-R-X-X-K-N), cyclo(M-K-X-D-H-R-X-X-X-N), and cyclo(M-X-X-S-X-X-R-W-X-T-X-N).

26. A library comprising members based upon an amino acid sequence motif for an interaction site of a binding compound for CC chemokine receptor 5, the motif being determined by permitting at least one peptide member from a peptide library to interact with said binding compound for CC chemokine receptor 5, and determining an amino acid sequence of at least one peptide that interacts with said binding compound for CC chemokine receptor 5.

27. A method of solubilizing and immobilizing a compound corresponding to the binding property of CC chemokine receptor 5, wherein the solubilization and immobilization is conducted substantially in the absence of sodium chloride when determining a compound corresponding to the binding of CC chemokine receptor 5.

28. A method of solubilizing and immobilizing a compound corresponding to the binding property of CC chemokine receptor 5, wherein the solubilization and immobilization is conducted by using a low salt concentration when determining a compound corresponding to the binding of CC chemokine receptor 5.

29. The method of claim 28, wherein said low salt concentration comprises a pre-determined amount magnesium and calcium.

30. A CC chemokine receptor 5 transfer vector constructed wherein said transfer vector comprises a CC chemokine receptor 5 molecule and a tag selected from the group consisting of GST, FLAG, 6xHis, C-MYC, MBP, V5, Xpress, CBP, and HA.

31. A method of using three-dimensional structure of CC chemokine receptor 5 in a drug screening assay comprising:
a) selecting a potential drug by performing rational drug design with the three-dimensional structure, wherein said selecting step is performed in conjunction with computer modeling;
b) contacting the potential drug with a first molecule comprising a first CC chemokine receptor 5; and
c) detecting the binding of the potential drug with said first molecule; wherein a potential drug is selected as a drug if the potential drug binds to said first molecule.

32. The method of claim 31, wherein said first molecule is labeled.

33. The method of claim 31, wherein said first molecule is bound to a solid support.
